SEQUENCE LISTING (7 sequences)

SEQ ID NO: 1 - protein, 605 aa, Artificial Sequence
  Description of Artificial Sequence Synthetic polypeptide
MQGPHLSLLLLLLATMGAVLQADTVEELTNTRGLIFLEDGVWPPSSPPEPLCLVAVRGEGDTSKASLTVVGGLHSYEHAFLEAVQESRWGPQDLATFGVCSTDSQTTLPALQRLGAWLGETGEQQLLVLHLAEVIWEPQLLLKFQEPPPGGASRWEQALLVLYPGPGPQVTVTGAGLQGTQSLCPTRDTRYLVLTVHFPAGAWSGSGLALTLQPSKEGATLTIAQLQAFLFGSDSRCFTRMTPTLVLLPPTGPTPQPAHGQLDTVPFPQPGLSLEPEDLPHSADPFLETLTRLVRALRGPLTRASNTRLALDPGALASFPQGLVNLSDPVALGRLLDGEEPLLLLLSPAAATVGEPMRLHSPTSAPWAAGLARRVAVELQAAASELRDLPGLPPTAPPLLSRLLALCPNDSRSAGDPLRALLLLKALQGLRAEWRGREGRGSNATGSGSNATSRSGSNATGSGSNATSFEGKPIPNPLLGLDSTRTGHHHHHHRARRSKGTGTDGLCALRELSVDLRAERSVLIPETYQANNCQGACAWPQSDRNPRYGNHVVLLLKMQARGAALGRLPCCVPTAYTGKLLISLSEEHISAHHVPNMVATECGCR

SEQ ID NO: 2 - DNA, 2065 bp, Homo sapiens
gcatgttgacacatcaggcccagctctatcactggggagggagataggctgccagggacagaaagggctctttgagaaggccactctgcctggagtgggggcgccgggcactgtcccccaaggtcgcggcagaggagataggggtctgtcctgcacaaacaccccaccttccactcggctcacttaaggcaggcagcccagcccctggcagcacccacgatgcgggacctgcctctcaccagcctggccctagtgctgtctgccctgggggctctgctggggactgaggccctcagagcagaggagccagctgtgggcaccagtggcctcatcttccgagaagacttggactggcctccaggcagcccacaagagcctctgtgcctggtggcactgggcggggacagcaatggcagcagctcccccctgcgggtggtgggggctctaagcgcctatgagcaggccttcctgggggccgtgcagagggcccgctggggcccccgagacctggccaccttcggggtctgcaacaccggtgacaggcaggctgccttgccctctctacggcggctgggggcctggctgcgggaccctggggggcagcgcctggtggtcctacacctggaggaagtgacctgggagccaacaccctcgctgaggttccaggagcccccgcctggaggagctggccccccagagctggcgctgctggtgctgtaccctgggcctggccctgaggtcactgtgacgagggctgggctgccgggtgcccagagcctctgcccctcccgagacacccgctacctggtgttagcggtggaccgccctgcgggggcctggcgcggctccgggctggccttgaccctgcagccccgcggagaggactcccggctgagtaccgcccggctgcaggcactgctgttcggcgacgaccaccgctgcttcacacggatgaccccggccctgctcctgctgccgcggtccgagcccgcgccgctgcctgcgcacggccagctggacaccgtgcccttcccgccgcccaggccatccgcggaactcgaggagtcgccacccagcgcagaccccttcctggagacgctcacgcgcctggtgcgggcgctgcgggtccccccggcccgggcctccgcgccgcgcctggccctggatccggacgcgctggccggcttcccgcagggcctagtcaacctgtcggaccccgcggcgctggagcgcctactcgacggcgaggagccgctgctgctgctgctgaggcccactgcggccaccaccggggatcctgcgcccctgcacgaccccacgtcggcgccgtgggccacggccctggcgcgccgcgtggctgctgaactgcaagcggcggctgccgagctgcgaagcctcccgggtctgcctccggccacagccccgctgctggcgcgcctgctcgcgctctgcccaggtggccccggcggcctcggcgatcccctgcgagcgctgctgctcctgaaggcgctgcagggcctgcgcgtggagtggcgcgggcgggatccgcgcgggccgggtcgggcacagcgcagcgcgggggccaccgccgccgacgggccgtgcgcgctgcgcgagctcagcgtagacctccgcgccgagcgctccgtactcatccccgagacctaccaggccaacaattgccagggcgtgtgcggctggcctcagtccgaccgcaacccgcgctacggcaaccacgtggtgctgctgctgaagatgcaggcccgtggggccgccctggcgcgcccaccctgctgcgtgcccaccgcctacgcgggcaagctgctcatcagcctgtcggaggagcgcatcagcgcgcaccacgtgcccaacatggtggccaccgagtgtggctgccggtgacccctgcgccgcgcggactcctgccccgagggtccggacgcgccccagctcgcgccccttcccatatttattcggaccccaagcatcgccccaataaagaccagcaagcaaccggcaaaaaaaaaaaaaaaaaaaaaaaaaaaaaaaaaaaaaaaaaaaaaaa

SEQ ID NO: 3 - protein, 560 aa, Homo sapiens
MRDLPLTSLALVLSALGALLGTEALRAEEPAVGTSGLIFREDLDWPPGIPQEPLCLVALGGDSNGSSSPLRVVGALSAYEQAFLGAVQRARWGPRDLATFGVCNTGDRQAALPSLRRLGAWLRDPGGQRLVVLHLEEVTWEPTPSLRFQEPPPGGAGPPELALLVLYPGPGPEVTVTRAGLPGAQSLCPSRDTRYLVLAVDRPAGAWRGSGLALTLQPRGEDSRLSTARLQALLFGDDHRCFTRMTPALLLLPRSEPAPLPAHGQLDTVPFPPPRPSAELEESPPSADPFLETLTRLVRALRVPPARASAPRLALDPDALAGFPQGLVNLSDPAALERLLDGEEPLLLLLRPTAATTGDPAPLHDPTSAPWATALARRVAAELQAAAAELRSLPGLPPATAPLLARLLALCPGGPGGLGDPLRALLLLKALQGLRVEWRGRDPRGPGRAQRSAGATAADGPCALRELSVDLRAERSVLIPETYQANNCQGVCGWPQSDRNPRYGNHVVLLLKMQVRGAALARPPCCVPTAYAGKLLISLSEERISAHHVPNMVATECGCR

SEQ ID NO: 4 - protein, 14 aa, Artificial Sequence
  Description of Artificial Sequence Synthetic peptide
GKPIPNPLLGLDST

SEQ ID NO: 5 - protein, 6 aa, Artificial Sequence
  Description of Artificial Sequence Synthetic 6xHis tag
HHHHHH

SEQ ID NO: 6 - protein, 108 aa, Artificial Sequence
  Description of Artificial Sequence Synthetic polypeptide
SKGTGTDGLCALRELSVDLRAERSVLIPETYQANNCQGACAWPQSDRNPRYGNHVVLLLKMQARGAALGRLPCCVPTAYTGKLLISLSEEHISAHHVPNMVATECGCR

SEQ ID NO: 7 - protein, 109 aa, Homo sapiens
SAGATAADGPCALRELSVDLRAERSVLIPETYQANNCQGVCGWPQSDRNPRYGNHVVLLLKMQVRGAALARPPCCVPTAYAGKLLISLSEERISAHHVPNMVATECGCR

(19) United States
(12) Patent Application Publication
(10) Pub. No.: US 2012/0225824 A1
(43) Pub. Date: Sep. 6, 2012

(54) MULLERIAN INHIBITING SUBSTANCE (MIS) ANALOGUES

(75) Inventors: Joyce W. Lustbader, Demarest, NJ (US); Drew Tortoriello, Jersey City, NY (US)

(73) Assignee: THE TRUSTEES OF COLUMBIA UNIVERSITY IN THE CITY OF NEW YORK, New York, NY (US)

(21) Appl. No.: 13/057,674

(22) PCT Filed: Aug. 5, 2009
(86) PCT No.: PCT/US09/52842

§ 371 Date: Sep. 22, 2011

Related U.S. Application Data
(60) Provisional application No. 61/137,948, filed on Aug. 5, 2008.

(51) Int. Cl. A61K 38/17; A61P 15/00; C12N 5/02; C07K 14/435

(52) U.S. Cl. 514/18.9; 530/350; 435/375; 435/366

(57) ABSTRACT

Mullerian Inhibiting Substance analogues are disclosed which comprise one or more non-naturally occurring N-linked glycosylation sites, protease cleavage sites, and/or tags such as an epitope tag. Also disclosed are pharmaceutical compositions comprising such compositions and methods of using such compositions, for example in the treatment of conditions associated with uncontrolled growth of a tissue derived from a Mullerian duct, such as the condition endometriosis.

This application claims the benefit of the filing date of U.S. Provisional Patent Application No. 61/137,948 filed Aug. 5, 2008.

For the purpose of the U.S. and other PCT contracting states that permit incorporation by reference only, all patents, patent applications and publications cited herein are hereby incorporated by reference in their entirety.

The invention disclosed herein was made with government support under Grant No. K12HD01275 from the National Institute of Child Health and Human Development. Accordingly, the U.S. Government has certain rights in this invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND

Endometriosis, which is characterized by the growth of functional endometrial tissue outside the uterine cavity, frequently causes dysmenorrhea, dyspareunia, chronic pelvic pain and/or infertility. Its prevalence is estimated at 10% of all reproductive-age women (Eskenazi B, Warner M L 1997 Epidemiology of endometriosis. Obstet Gynecol Clin North Am 24:235-258), but has been found in 12 to 32% of reproductive age women undergoing laparoscopy to determine the cause of pelvic pain, 9 to 50% of women undergoing laparoscopy for infertility, and 50% of teenagers undergoing laparoscopy for evaluation of chronic pelvic pain or dysmenorrhea. These symptoms increase work absenteeism, create social isolation, deplete financial resources and contribute to an overall deterioration in a woman's quality of life.

One treatment of pelvic pain associated with endometriosis includes surgical extirpation of endometriotic lesions with medical treatments to lower estrogen levels. In a prospective, randomized, double-blind trial comparing laser ablation of endometriotic lesions and laparoscopic nerve ablation to diagnostic laparoscopy with expectant management, pain symptoms were significantly improved in patients receiving laser ablation (Sutton C J, Ewen S P, Whitelaw N, Haines P 1994 Prospective, randomized, double-blind, controlled trial of laser laparoscopy in the treatment of pelvic pain associated with minimal, mild, and moderate endometriosis. Fertil Steril 62:696-700). However, only 55% of these patients maintained satisfactory symptomatic relief at 72 months following the surgery. Thus, hormonal treatments such as gonadotropin releasing hormone agonists (GnRHa), progestins, combined oral contraceptive pills, and danazol are often utilized for long-term management of the disease.

Although these medications provide significantly more pain relief than placebo, 30-60% of treated patients do not achieve relief for more than six months (Howard F M 2000 An evidence-based medicine approach to the treatment of endometriosis-associated chronic pelvic pain: placebo-controlled studies. J Am Assoc Gynecol Laparosc 7:477-488). Furthermore, danazol has poor tolerability due to androgenic side effects, and GnRHa tend to induce unpleasant menopausal symptoms such as hot flushes, vaginal dryness and loss of libido. Moreover, use of GnRHa for more than six months fosters significant bone mineral density loss from hypoestrogegemia. While partial estrogen replacement successfully reduces this bone loss, they increase the complexity and cost of treatment and may attenuate the therapeutic effect of hypoestrogenism on endometriotic lesions (Hornstein M D, Surrey E S, Weisberg G W, Casino L A 1998 Leuprolide acetate depot and hormonal add-back in endometriosis: a 12-month study. Lupron Add-Back Study Group. Obstet Gynecol 91:16-24).

Additional medical therapies include non-steroidal anti-inflammatory drugs (NSAIDs) and aromatase inhibitors. In a double-blind, cross-over study, naproxen, an NSAID, provided significantly greater relief of secondary dysmenorrhea associated with endometriosis compared to that of placebo (Kauppila A, Ronnberg L 1985 Naproxen sodium in dysmenorrhea secondary to endometriosis. Obstet Gynecol 65:379-383). However, gastric irritation usually limits long-term NSAID use. While aromatase inhibitors target the abnormal expression of the enzyme in ectopic endometrial tissue, they also profoundly suppress ovarian estrogen production resulting in the need for add-back therapy, similar to GnRHa, to prevent bone loss (Takayama K, Zeitoun K, Gunby R T, Sasano H, Carr B R, Bulun S E 1998 Treatment of severe postmenopausal endometriosis with an aromatase inhibitor. Fertil Steril 69:709-713).

It is clear that none of the currently available medical or surgical therapies provide optimal long-term treatment. Furthermore, women with endometriosis desiring pregnancy essentially have no medical treatment options since conception is generally not possible during hormonal therapy.

### SUMMARY OF THE INVENTION

The present invention provides Müllerian Inhibiting Substance (MIS) analogues that comprise one or more non-naturally occurring N-linked glycosylation sites, protease cleavage sites, and/or tags such as epitope tags. The present invention also provides pharmaceutical compositions that comprise or consist essentially of such compositions, and methods of using such compositions, for example in the treatment of conditions associated with uncontrolled growth of a tissue derived from a Müllerian duct.

In one embodiment, the present invention provides a MIS analogue comprising one or more non-naturally occurring N-linked glycosylation recognition sequences and an approximately 108-109 amino acid C-terminal fragment which can be released from the MIS analogue by proteolytic cleavage. In preferred embodiments, the N-linked glycosylation recognition sequences are not located within the C-terminal fragment but are instead located N-terminal to the C-terminal fragment. In preferred embodiments the MIS analogues of the invention comprise two to six N-linked glycosylation recognition sequences. In further preferred embodiments, the MIS analogue comprises a cluster of two to six N-linked glycosylation recognition sequences within the 100 or 80 or 60 or 40 or 20 amino acids N-terminal to the to the C-terminal fragment of MIS.

In other embodiments, the MIS analogues of the invention comprise one or more non-naturally occurring protease cleavage sites located N-terminal to, at the site of, the start of the C-terminal fragment. In a preferred embodiment the protease cleavage site is a furin cleavage site.

In other embodiments, the MIS analogues of the invention comprise a tag sequence selected from the group consisting of myc, T7, GST, GFP, HA (hemagglutinin), V5, His, GST, and FLAG tags.

In certain preferred embodiments, the MIS analogue of the invention comprises a sequence that has about 90% or greater sequence identity with SEQ ID NO. 1, or consists essentially of a sequence that has about 90% or greater sequence identity with SEQ ID NO. 1, or consists of a sequence that has about 90% or greater sequence identity with SEQ ID NO. 1. In other preferred embodiments, the MIS analogue of the invention comprises SEQ ID NO. 1, or consists essentially of SEQ ID NO. 1, or consists of SEQ ID NO. 1.

The present invention also provides a pharmaceutical composition that comprises or consists essentially of the MIS analogues of the present invention.

In certain embodiments, the present invention provides a method for inhibiting proliferation of, or inducing apoptosis in, cells derived from the Müllerian duct, the method comprising contacting cells derived from the Müllerian duct with an effective amount of a MIS analogue of the invention. The cells may be in vitro or may alternatively be in a subject, preferably in a human female subject. The cells may be, for example, endometrial cells, other uterine cells, ovarian cells, cells of the oviduct, cervical cells, and vaginal cells.

The present invention also provides methods for treating conditions associated with uncontrolled growth of a tissue derived from a Müllerian duct in a subject, the methods comprising administering to the subject an effective amount of a MIS analogue of the invention. In preferred embodiment, the subject is a human female. In further preferred embodiments, the human female has, or is at risk of developing, a condition selected from the group consisting of endometriosis, adenomyosis, uterine fibroids, cancer of the cervix, cancer of the ovary, and cancer of the uterus. In particularly preferred embodiments, the present invention provides a method for treating endometriosis in a human female subject comprising administering to the subject an effective amount a MIS analogue of the invention.

These and other embodiments are described in the following description, claims, and drawings.

### BRIEF DESCRIPTION OF THE FIGURES

FIG. 1: Müllerian-inhibiting substance signaling genes are present in human endometrium. cDNA derived from a representative proliferative endometrium sample was polymerase chain reaction amplified for the following genes and conditions: lane 1) HPRT; lane 2) HPRT (no cDNA control); lane 3) HPRT (no reverse transcriptase control); lane 4) ALK3; lane 5) MISRII; lane 6) Smad 1; lane 7) MIS; and lane 8) Smad 9.

FIGS. 2A-2F: Müllerian-inhibiting substance (MIS) and MIS type II receptor (MISRII) protein are expressed in human endometrium. Fixed sections of human proliferative (A, D) and secretory (B, E) endometrium stained for MIS (A, D) and MISRII (B, E) revealed strong immunoreactivity for both proteins in the glandular cells. Stromal cell immunoreactivity for these proteins appeared to be increased in the secretory phase (B, E). Use of secondary antibody only as a negative control revealed no immunoreactivity (C, F). When the MIS antibody was preadsorbed with fivefold excess recombinant MIS, an absence of immunoreactivity was also noted.

FIG. 3: Transient transfection of MIS construct into CHO-K1 cells. Pro-hyper-MIS1 was identified at the expected molecular weight in the cell lysate fraction but not in the supernatant fraction. Western blot of MIS analogues isolated from cell culture fractions using anti-human MIS antibody. CL is the cell lysate fraction. S is the supernatant fraction. The hyper-pro MIS1 protein was isolated from the lysate confirming the protein was hyperglycosylated. Protein was not present in the supernatant fraction suggesting the hormone was not secreted. Recombinant human MIS was observed at MW 65 in both the lysate and supernatant fractions.

FIG. 4: Immunohistochemistry indicates that MIS and MISRII proteins are produced but are primarily restricted to cells undergoing mitosis. Immunohistochemistry of MIS expression in cultured endometrial cells. MIS protein was identified primarily in cells undergoing mitosis. The two small arrows identify a cell in early telophase of mitosis. The large arrow identifies a cell late in telophase undergoing cytokinesis.

FIG. 5: Amino acid sequence of an exemplary MIS analogue (SEQ ID NO. 1). This sequence is based on the sequence of the human MIS polypeptide (SEQ ID NO. 3) but has been modified to include various changes to the amino acid sequence, including to incorporate N-linked glycosylation sequences, a V5 epitope tag, a hexahistidine tag, and a furin cleavage site. The amino acids that constitute the 108 amino acid C-terminal fragment or “active peptide” of the MIS analogue are also indicated in bold type face. This 108 amino acid sequence represented in bold type face is SEQ ID NO. 6.

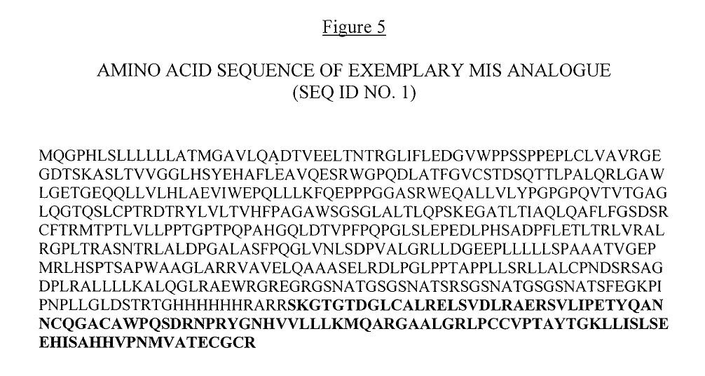

FIG. 6: Schematic Representation of the structure of the MIS analogue of SEQ ID NO. 1, indicating the relative positions of the N-linked glycosylation sequences, the V5 epitope tag, the hexahistidine tag, and the furin cleavage site. The location of the 108 amino acid C-terminal fragment or “active peptide” of the MIS analogue are indicated.

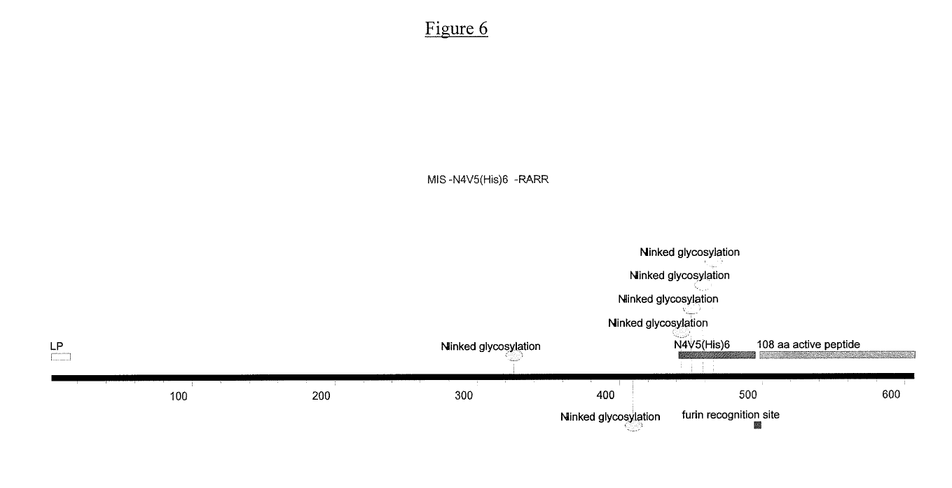

FIG. 7: Nucleotide sequence (SEQ ID NO. 2, GenBank accession no. NM_{—}000479) which encodes a human MIS polypeptide. This nucleotide sequence has not been modified as provided by the present invention and does not encode an MIS analogue of the invention.

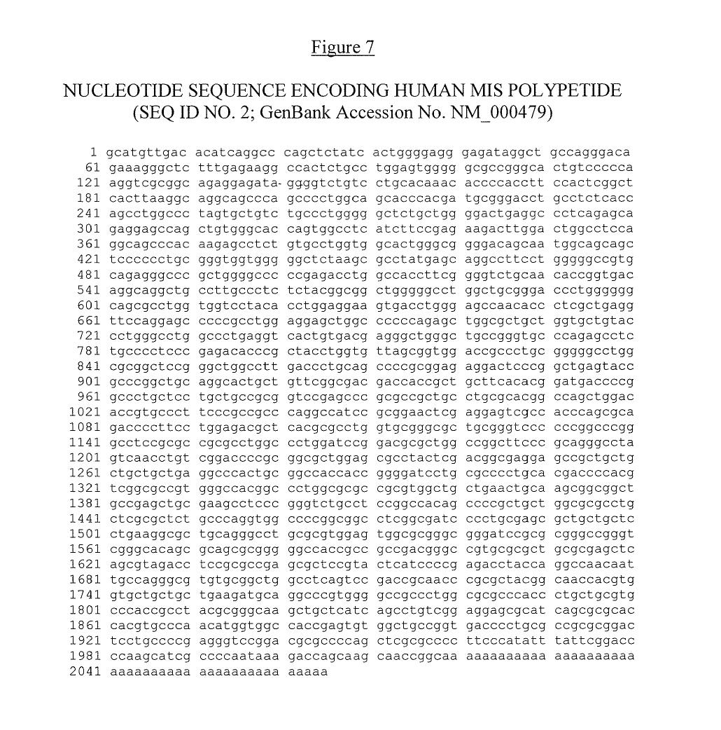

FIG. 8: Amino acid sequence of a human MIS polypeptide (SEQ ID NO. 3, GenBank accession no. P03971). This amino acid sequence has not been modified according as provided by the present invention and is not an MIS analogue of the invention. The amino acids that comprise the 109 C-terminal fragment of the MIS polypeptide are shown in bold type face. This 109 amino acid sequence represented in bold type face is SEQ ID NO. 7.

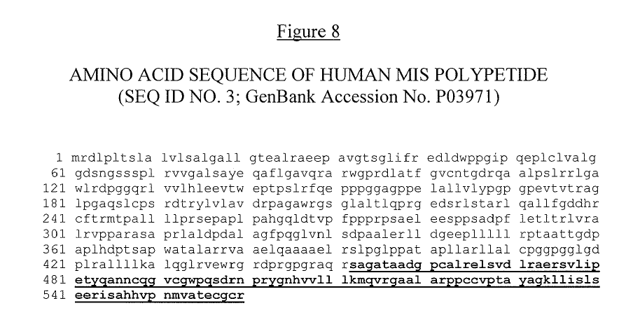

### DETAILED DESCRIPTION

The present invention provides Müllerian Inhibiting Substance analogues, compositions comprising such analogues, and methods of use of such analogues, for example in the treatment of endometriosis.

Müllerian Inhibiting Substance Analogues

Müllerian Inhibiting Substance or “MIS” is also known in the art as Anti-Müllerian Hormone (AMH), Müllerian Inhibiting Factor (MIF), and Müllerian Inhibiting Hormone (MIH). MIS is a 140 kDa glycosylated disulfide-linked homodimeric protein of the TGF-β superfamily. Under reducing conditions MIS migrates on an electrophoretic gel at an apparent molecular weight of around 70 kDa. The MIS protein can be proteolytically cleaved, for example by plasmin, to produce two distinct fragments. The C-terminal fragment is about 109 amino acids long and has a molecular weight of about 25 kDa. The N-terminal fragment has a molecular weight of about 57 kDa. The biological activity of MIS is believed to be primarily attributable to this C-terminal fragment. However, there is some evidence in the art that both the N-terminal and C-terminal fragments have biological activity and that the N- and C-terminal fragments remain associated in a non-covalent complex.

MIS is known to cause regression of tissues of the Müllerian duct during development of male embryos. It is also known that MIS can exert both anti-proliferative and pro-apoptotic effects on cells and tissues derived from the Müllerian duct. Tissues that are derived from the Müllerian ducts include, but are not limited to, tissues of the uterus, including the endometrium, tissues of the upper vagina, tissues of the cervix, ovarian tissues, and tissue of the oviducts. Various conditions are associated with excessive and/or uncontrolled growth of a tissue derived from a Müllerian duct including, but not limited to, endometriosis, adenomyosis, uterine fibroids, cancer of the cervix, cancer of the ovary, and cancer of the uterus.

The Müllerian Inhibiting Substance (MIS) analogues of the present invention contain modifications that are not found in naturally occurring MIS polypeptides. For example, the MIS analogues of the invention may comprise one or more non-naturally occurring N-linked glycosylation recognition sequences, non-naturally occurring protease recognition sites, and/or non-naturally occurring antigenic tags. These modifications result in improved properties of the MIS analogues of the present invention as compared to naturally occurring MIS polypeptides. For example, the non-naturally occurring N-linked glycosylation recognition sequences incorporated into the MIS analogues of the invention may increase the half-life of the MIS analogues.

The MIS analogues of the invention may be based on the amino acid sequence of any MIS polypeptide, such as any mammalian MIS polypeptide. For example, the MIS analogue of the invention may be based on the sequence of a human, mouse, rat, bovine, or canine, or porcine MIS polypeptide. Sequences of suitable MIS polypeptides include those having GenBank accession numbers: CAA01397 (human), NP_{—}000470 (human), AAC25614 (human), P03971 (human), AAA98805 (human), AAV97596 (dog), AAA20970 (mouse), P27106 (mouse), NP_{—}031471 (mouse), AAW84293 (mouse), AAB22104 (Rat), P49000 (rat), NP_{—}037034 (rat) P03972 (bovine), NP_{—}776315 (bovine), AAA98765 (bovine), and P79295 (pig), and those amino acid sequences described in U.S. Pat. Nos. 5,047,336 and 5,661,126, the contents of which are hereby incorporated by reference. In addition, the MIS analogues of the invention may be encoded by a nucleotide sequence that is based on any MIS-encoding nucleotide sequence, such as any mammalian MIS-encoding nucleotide sequence. Sequences of suitable MIS-encoding nucleotides include those having GenBank accession numbers: NG_{—}012190 (human), NM_{—}000479 (human), K03474 (human), AY911505 (mouse), U09208 (mouse), NM_{—}007445 (mouse), NM_{—}012902 (rat), S98336 (rat), A18521, E01304, AY673990 (dog), M13151 (bovine), and NM_{—}173890 (bovine), and those nucleotide sequences described in U.S. Pat. Nos. 5,047,336 and 5,661,126, the contents of which are hereby incorporated by reference.

The MIS analogues of the present invention may be based on any of the MIS polypeptide sequences listed above, or sequences that have about 70% or more, or about 75% or more, or about 80% or more, or about 85% or more, or about 90% or more, or about 91%, 92%, 93%, 94%, 95%, 96%, 97%, 98%, or 99% or more amino acid identity with any of the MIS polypeptide sequences listed above. In addition, the MIS analogues may be based on MIS polypeptides that have been altered in some useful way, such as by including a fusion protein, by adding, removing, or substituting certain amino acids (for example to increase stability of activity), by conjugating the MIS polypeptide with other substances, e.g. polyethylene glycol, and the like. All such variant forms of MIS that retain one or more activities of MIS are within the scope of the present invention and can be used as the basis of the MIS analogs described herein.

In preferred embodiments, the MIS analogues of the present invention are based on one or more of the MIS polypeptide sequences described above and are modified to comprise one or more non-naturally occurring N-linked glycosylation recognition sequences, non-naturally occurring protease recognition sites, and/or non-naturally occurring antigenic tags. In preferred embodiments, the MIS analogues of the invention comprise one or more non-naturally occurring N-linked glycosylation recognition sequences and a non-naturally occurring protease recognition site. In further preferred embodiments, the MIS analogues of the invention comprise one or more non-naturally occurring N-linked glycosylation recognition sequences and a non-naturally occurring protease recognition site and a non-naturally occurring antigenic tag.

### N-Linked Glycosylation Recognition Sequences

The present invention relates, in part, to modified forms of MIS that comprise one or more non-naturally occurring N-linked glycosylation recognition sequences. Such N-linked glycosylation recognition sequences may increase the half-life of the MIS protein. The phrase “non-naturally occurring,” as used herein, refers to N-linked glycosylation recognition sequences are not found in the amino acid sequence of naturally occurring MIS polypeptides or that are not found in the amino acid sequence of naturally occurring MIS polypeptides at the specific location where they occur in the MIS analogues of the invention. The N-linked glycosylation recognition sequences used in accordance with the present invention preferably comprise the consensus sequence Asn-X-Ser or Asn-X-Thr or Asn-X-Cys, wherein X is any amino acid except proline, and wherein the peptide containing such sequences are capable of being glycosylated at the Asn residue of the recognition sequence. Such N-linked glycosylation sequences may be added to the sequence of any naturally occurring or other MIS polypeptide.

The MIS analogues of the present invention may comprise one, two, three, four, five, six, seven, eight, nine, ten or more non-naturally occurring N-linked glycosylation recognition sequences. In one preferred embodiment, the MIS analogues of the present invention comprise four non-naturally occurring N-linked glycosylation recognition sequences. In another preferred embodiment, the MIS analogues of the present invention comprise five non-naturally occurring N-linked glycosylation recognition sequences. In another preferred embodiment, the MIS analogues of the present invention comprise six non-naturally occurring N-linked glycosylation recognition sequences. SEQ ID NO. 1 illustrates an exemplary MIS analogue of the present invention comprising multiple non-naturally occurring N-linked glycosylation recognition sequences.

In preferred embodiments, at least some of the N-linked glycosylation recognition sequences are “clustered”, for example, such that one N-linked glycosylation site within the cluster is no more than about four amino acids apart from the next, or no more than about six amino acids apart from the next, or no more than about eight amino acids apart from the next, or no more than about ten amino acids apart from the next, or no more than about twelve amino acids apart from the next, or no more than about fourteen amino acids apart from the next, or no more than about sixteen amino acids apart from the next, or no more than about eighteen amino acids apart from the next, or no more than about twenty amino acids apart from the next. SEQ ID NO. 1 illustrates an exemplary MIS analogue comprising a cluster of four N-linked glycosylation sites within a twenty-two amino acid region wherein the Asn residue of each N-linked glycosylation recognition sequence is no more than 8 amino acid residues from the next such residue.

In a further preferred embodiment, the MIS analogues of the present invention comprise a cluster of N-linked glycosylation sites that is located N-terminal to the sequence that will ultimately be cleaved-off to form the active C-terminal fragment of MIS. For example, the MIS analogues of the present invention may comprise a cluster of N-linked glycosylation sites located within the 100 amino acids N-terminal to the to the C-terminal fragment of MIS, or the 80 amino acids located adjacent and N-terminal to the to the C-terminal fragment of MIS, or the 60 amino acids located adjacent and N-terminal to the to the C-terminal fragment of MIS, or the 40 amino acids located adjacent and N-terminal to the to the C-terminal fragment of MIS, or the 20 amino acids located adjacent and N-terminal to the to the C-terminal fragment of MIS, SEQ ID NO. 1 illustrates an exemplary MIS analogue comprising a cluster of four N-linked glycosylation sites located within the 100 amino acids located adjacent and N-terminal to the to the C-terminal fragment of MIS.

One of skill in the art can readily engineer a nucleotide sequence that encodes MIS to include the N-linked glycosylation sites described herein using standard molecular biology techniques, such as those described, for example, in Sambrook et al. (2001) Molecular Cloning: A Laboratory Manual, 3rd Ed., Cold Spring Harbor Laboratory, Cold Spring Harbor, N.Y. (“Sambrook”). For example, one of skill in the art can readily generate a nucleotide sequence that encodes an MIS analogue having such N-linked glycosylation sites by mutating any of the MIS-encoding nucleotide sequences described herein, such as a nucleotide sequence (e.g. SEQ ID NO. 2) that encodes the human MIS polypeptide (e.g. SEQ ID NO. 3) to introduce the desired modifications. Any suitable method to introduce mutations may be employed, including, but not limited to, PCR-based methods and site-directed mutagenesis methods.

### Protease Cleavage Sites

As described above, MIS polypeptides are typically proteolytically cleaved to produce two distinct fragments, including the approximately 108-109 amino acid C-terminal fragment to which the biological activity of MIS is primarily attributable. In some embodiments the present invention provides MIS analogues that comprise one or more non-naturally occurring protease recognition/cleavage sites. The phrase “non-naturally occurring,” as used in the context of protease recognition/cleavage sites, refers to protease recognition sites that are not found in the amino acid sequence of naturally occurring MIS polypeptides or not found in the same location in a naturally occurring MIS polypeptides.

In a preferred embodiment, the protease recognition/cleavage site may be located at a suitable position in the MIS analogue to enable proteolytic cleavage of the active C-terminal fragment of MIS. The exact placement of the protease recognition site may vary depending on the exact sequence of the protease recognition site, the manner in which the protease cleaves that recognition sequence, and the location of the C-terminal fragment of MIS within the whole MIS polypeptide. One of skill in the art can select the optimal location for placement of the protease recognition site. Typically the protease recognition site will be placed such that the protease that recognizes that sequence will cleave the MIS polypeptide at a location 108-109 amino acids N-terminal to the C-terminus of the MIS polypeptide, thereby generating a 108-109 amino acid C-terminal fragment of MIS. In various embodiments, the proteolytic cleavage site is located within the approximately 30, or approximately 25, or approximately 20, or approximately 15, or approximately 10, or approximately 5, or approximately 2 amino acid residues N-terminal to the start of the C-terminal fragment. The proteolytic cleavage site may also be located at the precise location of the start of the C-terminal fragment. One of skill in the art can readily determine the location of the start of the C-terminal fragment in any MIS polypeptide by reference to the literature and sequence databases, by performing sequence alignments and comparisons, such as by performing comparisons to the sequences disclosed herein, and/or by determining the sequence of the N-terminal end of the released 108-109 C-terminal fragment, and/or by any other suitable means.

Any suitable protease recognition site can be used. In one preferred embodiment, the skilled artisan will select a protease recognition site that is recognized by a protease that is expressed in the location to which the MIS analogue will be administered, such that the MIS analogue may be properly cleaved to generate the active C-terminal fragment at the desired location. In another preferred embodiment, the skilled artisan will select a protease recognition site that is recognized by a protease that is expressed in the cells to which the MIS analogue will be delivered. In another preferred embodiment, the skilled artisan will select a protease recognition site that is recognized by a protease that can be exogenously delivered to the desired cells or tissues.

Suitable protease recognition sites include, but are not limited to, serine protease recognition sites and endopeptidase recognition sites. Amino acid sequences that comprise protease recognition sites are known in the art, and one of skill in the art can readily select a suitable sequence for inclusion in the MIS analogues of the invention. In one preferred embodiment, MIS analogues of the invention comprise a recognition site for a subtilisin-like proprotein convertase such as, for example, the subtilisin-like pro-protein convertase furin. The canonical furin recognition sequence is Arg-X-(Arg/Lys)-Arg, where X is any amino acid. SEQ ID NO. 1 illustrates an exemplary MIS analogue comprising a furin-protease cleavage site located at amino acid residues 494 to 497 located adjacent to and N-terminal to the to the C-terminal fragment of MIS.

One of skill in the art can readily engineer a nucleotide sequence that encodes MIS to include the protease recognition sites described herein using standard molecular biology techniques, such as those described, for example, in Sambrook et al. (2001) Molecular Cloning: A Laboratory Manual, 3rd Ed., Cold Spring Harbor Laboratory, Cold Spring Harbor, N.Y. (“Sambrook”). For example, one of skill in the art can readily generate a nucleotide sequence that encodes an MIS analogue having such protease recognition sites by mutating any of the MIS-encoding nucleotide sequences described herein, such as a nucleotide sequence (e.g. SEQ ID NO. 2) that encodes the human MIS polypeptide (e.g. SEQ ID NO. 3) to introduce the desired modifications. Any suitable method to introduce mutations may be employed, including, but not limited to, PCR-based methods and site-directed mutagenesis methods.

### Tag Sequences

In certain embodiments, the MIS analogues of the invention comprise one or more non-naturally occurring tag sequences. The term non-naturally occurring is used herein in the context of tag sequences to refer to amino acid sequences that are not present in a naturally occurring form of the MIS polypeptide.

In one embodiment, the MIS analogues of the present invention comprise one or more tags that can be used to facilitate detection and/or purification of the MIS analogue. Suitable tags include, but are not limited to the myc, T7, GST, GFP, HA (hemaglutinin), V5, His tags, GST tags, and FLAG epitope tags. Any suitable tag may be used, and one of skill in the art can readily select a suitable tag without undue experimentation. In one preferred embodiment, the V5 epitope tag is used to facilitate detection of the MIS analogues of the invention. In another preferred embodiment, a hexahistidine His tag is used. SEQ ID NO. 1 illustrates an exemplary MIS analogue comprising a V5 epitope tag (amino acid sequence GKPIPNPLLGLDST) at amino acid residues 471 to 484 and a hexahistidine tag (amino acid sequence HHHHHH) at amino acid residues 488 to 493. Tags may be used to facilitate detection and/or isolation/purification of the MIS analogues of the invention by any suitable means, including but not limited to, by immunohistochemistry, immunoprecipitation, flow cytometry, fluorescence detection, ELISA, immunoblotting (“western”), affinity chromatography, and the like.

One of skill in the art can readily engineer a nucleotide sequence that encodes MIS to include the tag sequences described herein using standard molecular biology techniques, such as those described, for example, in Sambrook et al. (2001) Molecular Cloning: A Laboratory Manual, 3rd Ed., Cold Spring Harbor Laboratory, Cold Spring Harbor, N.Y. (“Sambrook”). For example, one of skill in the art can readily generate a nucleotide sequence that encodes an MIS analogue having such a tag sequence by mutating any of the MIS-encoding nucleotide sequences described herein, such as a nucleotide sequence (e.g. SEQ ID NO. 2) that encodes the human MIS polypeptide (e.g. SEQ ID NO. 3) to introduce the desired modifications. Any suitable method to introduce mutations may be employed, including, but not limited to, PCR-based methods and site-directed mutagenesis methods.

### Exemplary MIS Analogues

In one preferred embodiment, the present invention provides an MIS analogue comprising multiple non-naturally occurring N-linked glycosylation sites, a furin-protease cleavage site, a hexahistidine antigenic tag and a V5 antigenic tag. The present invention provides an exemplary MIS analogue comprising each of these modifications and having the amino acid sequence:

(SEQ ID NO: 1)
MQGPHLSLLLLLLATMGAVLQADTVEELTNTRGLIFLEDGVWPPSSPPEPLCLVAVR
GEGDTSKASLTVVGGLHSYEHAFLEAVQESRWGPQDLATFGVCSTDSQTTLPALQRL
GAWLGETGEQQLLVLHLAEVIWEPQLLLKFQEPPPGGASRWEQALLVLYPGPGPQVT
VTGAGLQGTQSLCPTRDTRYLVLTVHFPAGAWSGSGLALTLQPSKEGATLTIAQLQA
FLFGSDSRCFTRMTPTLVLLPPTGPTPQPAHGQLDTVPFPQPGLSLEPEDLPHSADPFLE
TLTRLVRALRGPLTRASNTRLALDPGALASFPQGLVNLSDPVALGRLLDGEEPLLLLL
SPAAATVGEPMRLHSPTSAPWAAGLARRVAVELQAAASELRDLPGLPPTAPPLLSRL
LALCPNDSRSAGDPLRALLLLKALQGLRAEWRGREGRGSNATGSGSNATSRSGSNAT
GSGSNATSFEGKPIPNPLLGLDSTRTGHHHHHHRARRSKGTGTDGLCALRELSVDL
RAERSVLIPETYQANNCQGACAWPQSDRNPRYGNHVVLLLKMQARGAALGRLP
CCVPTAYTGKLLISLSEEHISAHHVPNMVATECGCR.

The 108 amino acids represented in bold type face in SEQ ID NO. 1 comprise the 108 amino acid C-terminal active fragment of this MIS analogue. This 108 amino acid sequence represented in bold type face is identified as SEQ ID NO. 6. The MIS analogue of SEQ ID NO. 1 was made by making certain modifications to the sequence of the human MIS polypeptide (SEQ ID NO. 3), for example, to include multiple non-naturally occurring N-linked glycosylation sites, a furin-protease cleavage site, a hexahistidine antigenic tag and a V5 antigenic tag. SEQ ID NO. 1 comprises N-linked glycosylation sites at amino acid residues 325, 409, 443, 450, 458, and 465, a V5 epitope tag (amino acid sequence GKPIPNPLLGLDST (SEQ ID NO. 4) at amino acid residues 471 to 484, a hexahistidine tag (amino acid sequence HHHHHH (SEQ ID NO. 5) at amino acid residues 488 to 493, and a furin cleavage site at amino acid residue 497. The MIS analogue of SEQ ID NO. 1 may be proteolytically cleaved to generate the 108 amino acid C-terminal fragment comprising residues 498-605, which is illustrated in bold type face in SEQ ID NO. 1.

In preferred embodiments, the present invention provides an MIS analogue that comprises or consists of the sequence of SEQ ID NO:1 or that has about, 90%, 91%, 92%, 93%, 94%, 95%, 96%, 97%, 98%, or 99% amino acid sequence identity with SEQ ID NO:1. In a preferred embodiment, the MIS analogue also retains one or more of the normal activities of MIS, such as for example, its anti-proliferative and/or pro-apoptotic activity on cells and tissues derived from the Müllerian duct, such as endometrial cells, other uterine cells, ovarian cells, oviduct cells, cervical cells, and vaginal cells.

### Methods of Making the MIS Analogues of the Invention

In certain embodiments, the MIS analogues of the invention are produced using standard methodologies useful for production of recombinant proteins. Such methods are well known to those of skill in the art. Methods suitable for preparing the MIS analogues of the invention using recombinant DNA techniques are provided by Cate, et al. (Cell 45:685-698 (1986) and by U.S. Pat. No. 5,047,336, the contents of both of which are hereby incorporated by reference. In preferred embodiments, a nucleotide encoding an MIS analogue of the invention is operatively linked to an expression control sequence in an expression vector, such that the MIS analogue will be produced when the expression vector is delivered to suitable cells. One of skill in the art can readily generate a nucleotide sequence encoding any of the MIS analogues described herein, for example by mutating any of the MIS-encoding nucleotide sequences described herein to introduce the desired modifications. Any suitable method to introduce such mutations may be employed, including, but not limited to, PCR-based methods and site-directed mutagenesis methods. Any suitable expression vector and any suitable expression control sequence may be used. The MIS analogues of the invention can be expressed in any cell type that is capable of glycosylating polypeptides on Asn residues. In preferred embodiments the MIS analogues of the invention are expressed in eukaryotic cells. In a more preferred embodiment, the MIS analogues of the invention are expressed in mammalian cells.

In preferred embodiments, the MIS analogues of the invention are isolated/purified prior to use. For example, the MIS analogues of the invention may be purified from the supernatant of cells that express MIS from an expression vector. Standard methods for purifying proteins may be used, including, but not limited to affinity chromatography methods. For example, the MIS analogues of the invention may be purified using the methods disclosed in U.S. Pat. No. 5,310,886 entitled “Purification of Müllerian Inhibiting Substance,” the contents of which are hereby incorporated by reference. In some embodiments, the MIS analogues of the invention may be purified using immunoaffinity chromatography methods employing antibodies that bind to the MIS analogue. Such antibodies may bind, for example, to MIS sequences or to one or more tags within the MIS analogue.

### Pharmaceutical Compositions

The MIS analogues of the present invention may be formulated as part of a pharmaceutical composition. In addition to one or more MIS analogues, such pharmaceutical compositions may comprise one or more pharmaceutically acceptable solvents (such as aqueous or non-aqueous solvents), diluents, carriers, vehicles, excipients, surfactants, adjuvants, preservatives, stabilizers, wetting agents, emulsifying agents, antibacterial agents, antifungal agents, sugars, salts, agents that promote sustained release of the active compounds, agents that facilitate or limit absorption, and the like. One of skill in the art can readily select suitable agents for inclusion in the pharmaceutical compositions of the invention, for example by consulting “Remington's Pharmaceutical Sciences”, Gennaro, A. R., 18th Edition, Mack Publishing Co., Easton, Pa.

The pharmaceutical compositions of the invention may also include one or more therapeutically effective agents in addition to the MIS analogues of the present invention, such as other agents that may be useful for treating a condition associated with excessive and/or uncontrolled growth of a tissue derived from a Müllerian duct.

### Methods of Use of the MIS Analogues of the Invention

In certain aspects, the present invention provides methods useful for the treatment of conditions associated with excessive and/or uncontrolled growth of a tissue derived from a Müllerian duct comprising administering to a subject a therapeutically effective amount of a MIS analogue of the invention. Tissues that are derived from the Müllerian ducts include, but are not limited to, tissues of the uterus, including the endometrium, tissues of the upper vagina, tissues of the cervix, ovarian tissues, and tissue of the oviducts. Conditions associated with excessive and/or uncontrolled growth of a tissue derived from a Müllerian duct include, but are not limited to, endometriosis, adenomyosis, uterine fibroids, cancer of the cervix, cancer of the ovary, and cancer of the uterus.

As used herein, the phrase “therapeutically effective amount” means an amount of the MIS analogue sufficient have an anti-proliferative and/or pro-apoptotic effect on cells and tissues derived from the Müllerian duct, such as endometrial cells, other uterine cells, ovarian cells, oviduct cells, cervical cells, and vaginal cells. The amount of the MIS analogues of the invention that should be used can be determined by routine experimentation and optimization. For example, effective amounts can be determined by performing in vitro experiments, in vivo animal experiments, and/or clinical trials, to quantify the effect of the MIS analogues of the invention on one or more parameters, such as to quantify its anti-proliferative and/or pro-apoptotic effect on cells and tissues derived from the Müllerian duct, such as endometrial cells, other uterine cells, ovarian cells, oviduct cells, cervical cells, and vaginal cells. The amount of the MIS analogue may then be altered accordingly to achieve the desired outcome. The therapeutically effective amount will depend upon a variety of factors including the disorder being treated and the severity of the disorder; the activity level of the MIS analogues employed, other components of the composition employed, the age and body weight of the subject, the time of administration, the route of administration, the duration of the treatment, drugs used in combination with or coincidental with administration of the MIS analogues of the invention, and other similar factors well known in the medical and pharmaceutical arts. For example, it is well within the skill of the art to start doses of the MIS analogues of the invention at levels lower than required to achieve the desired therapeutic effect and to gradually increase the dosage until the desired effect is achieved.

The MIS analogues of the invention may be administered by any suitable route, including by systemic or local administration. Contemplated delivery routes include, but are not limited to, intravenous delivery, intra-arterial delivery, intransal delivery subcutaneous delivery, transdermal delivery, oral delivery, and any other suitable delivery route known in the art. In preferred embodiments, the MIS analogues of the invention may be administered locally to the desired site of action, such as to the uterus, including the endometrial layer of the uterus, to the cervix, or to the ovaries. The MIS analogues can be administered locally by local injection, or by implantation of a preparation (such as a slow or sustained release hydrogel, a suppository, or a cream) or a device (such as a drug-releasing pump or a drug-coated intrauterine or intravaginal device) that comprises an MIS analogue at the desired site. Any method known in the art suitable for local delivery of a therapeutic agent to the uterus, cervix, or the ovaries may be used. In some embodiments, preparations and devices comprising the MIS analogues of the invention may be implanted locally at the desired site of action by a doctor without the need for anesthesia or surgery. In other embodiments, preparations and devices comprising the MIS analogues of the invention may be implanted during surgery. One of skill in the art can readily select a suitable means of administration of the MIS analogues of the invention without undue experimentation.

The subjects to be treated using the compositions and methods of the invention may be suffering from, or at risk of developing, a condition associated with excessive and/or uncontrolled growth of a tissue derived from a Müllerian duct. Such conditions include, but are not limited to, endometriosis, adenomyosis, uterine fibroids, cancer of the cervix, cancer of the ovary, and cancer of the uterus. Unless stated otherwise, the terms “treat” “treated” or “treatments” include curative methods, methods aimed at reducing the severity and/or duration of symptoms, methods of preventing symptoms, and methods of reducing the recurrence of symptoms. The subjects may be any human or other mammalian species. In preferred embodiments, the subjects are female humans. In even more preferred embodiments, the subjects are female humans that are afflicted with, have previously been afflicted with, or are at risk of developing endometriosis, adenomyosis, uterine fibroids, cancer of the cervix, cancer of the ovary, or cancer of the uterus.

In a preferred embodiment, the present invention provides a method of treating a disease in a subject is wherein a symptom of the disease is ectopic growth of a uterine tissue, comprising administering to the subject an amount of a MIS analogue of the invention effective to inhibit ectopic growth of the uterine tissue. As used herein, the term “uncontrolled growth” refers to abnormal or pathological growth. By way of a non-limiting example, such growth includes growth of endometrial tissue beyond or outside of the uterus or outside the endometrial layer of the uterus.

In another preferred embodiment, the present invention provides a method of treating endometriosis in a subject is comprising administering to the subject an amount of a MIS analogue of the invention effective to inhibit the endometriosis. Also contemplated by the present invention are methods useful for the treatment of conditions associated with excessive and/or uncontrolled growth of a tissue derived from a Müllerian duct comprising administering to a subject a therapeutically effective amount of nucleic acid molecule that encodes a MIS analogue of the invention, for example by administering a “naked” nucleic acid molecule or a vector that comprises the nucleic acid molecule. Using such methods it is contemplated that the MIS analogues of the invention will be expressed in vivo at the desired site of action. Site specific expression may be achieved by, for example, use of a tissue specific promoter, such as an endometrial specific promoter. One of skill in the art can readily select suitable vectors and promoters for expression of the MIS analogues of the invention in vivo.

In other aspects, the invention provides methods useful for the inhibiting the growth or proliferation of, or inducing or enhancing apoptosis in cells comprising contacting the cells with an effective amount of a MIS analogue of the present invention. In preferred embodiments the cells are endometrial cells, other uterine cells, cervical cells, ovarian cells, oviduct cells, or vaginal cells. In other preferred embodiments, the cells are derived from or within a subject afflicted with a condition associated with excessive and/or uncontrolled growth of a tissue derived from a Müllerian duct, such as endometriosis, adenomyosis, uterine fibroids, cancer of the cervix, cancer of the ovary, and cancer of the uterus. The cells may be located in a subject, such as a human female subject, or may be located in vitro, such as in tissue culture. Methods of administering the MIS analogues of the invention to cells in a subject are described above. The MIS analogues of the invention can be delivered to cells in vitro using any suitable method, for example, by direct addition of the MIS analogues of the invention, or a pharmaceutical composition comprising such MIS analogues, directly to a vessel containing such cells, or by using any other suitable deliver method known in the art, such as liposome mediated delivery and the like. Also contemplated are methods of delivering the MIS analogues of the invention to cells in vitro by delivering to the cells and effective amount of nucleic acid molecule that encodes a MIS analogue of the invention, for example by delivering a “naked” nucleic acid molecule or a vector that comprises the nucleic acid molecule, for example using standard transfection techniques. One of skill in the art can readily select a suitable transfection technique.

Also contemplated by the present invention are methods that comprise administration or delivery of a MIS analogue of the invention, or a nucleic acid that encodes a MIS analogue of the invention, in combination with, or co-incident with, or before, or after administration or delivery of a proteolytic enzyme. Such proteolytic enzymes are selected such that they are able to cleave the C-terminal fragment of the MIS analogue. Any suitable proteolytic enzyme may be used, such as any of those described above. In a preferred embodiment, the proteolytic enzyme is plasmin or furin. In preferred embodiments the protease is not contacted with the MIS analogue before the MIS analogue has been administered to its target location, such that the MIS analogue will remain intact and will only be cleaved by the protease to release its active form at the desired location. Thus, if the MIS analogue is to be administered with a protease, it is preferred that the protease either be administered to the desired site of action before or after the MIS analogue. In particularly preferred embodiments, no exogenous protease is delivered and instead an endogenous protease present at the desired site of action can cleave the MIS analogue to release the active C-terminal fragment.

### EXAMPLE

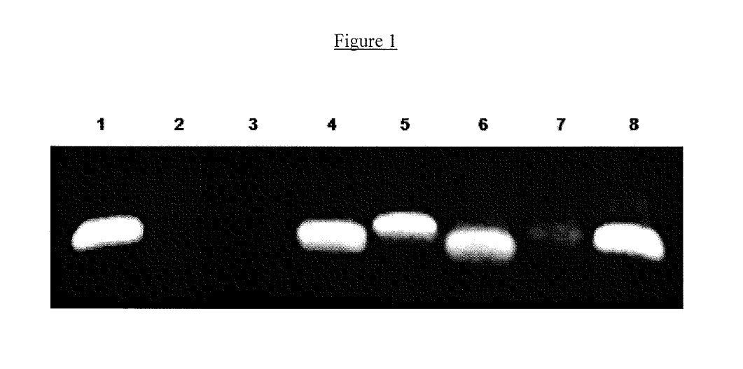

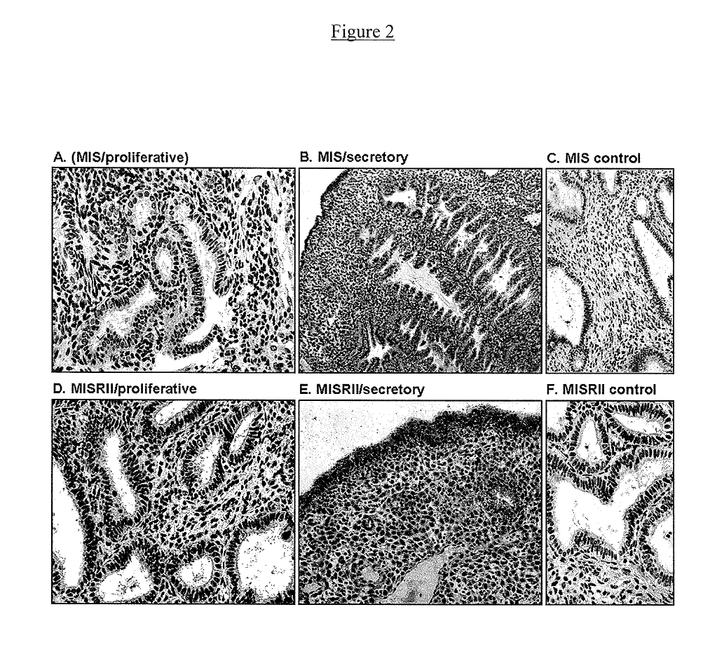

Müllerian inhibiting substance (MIS), originally named for its most well-described effect, namely the regression of the Müllerian duct in the developing male embryo, is secreted by the testicular Sertoli cells and fosters both anti-proliferative and pro-apoptotic effects in the adjacent Müllerian duct tissue in a paracrine fashion. MIS was investigated here as a treatment for endometrial tissue lesions (endometriosis), either eutopic or heterotopic. A series of experiments was performed to determine whether adult human eutopic and heterotopic endometrium possessed an intact MIS signaling system, and moreover, whether activation of this system could influence cell viability. FIG. 1. Shows Müllerian-inhibiting substance signaling genes are present in human endometrium. cDNA derived from a representative proliferative endometrium sample was polymerase chain reaction amplified for the following genes and conditions: lane 1) HPRT; lane 2) HPRT (no cDNA control); lane 3) HPRT (no reverse transcriptase control); lane 4) ALK3; lane 5) MISRII; lane 6) Smad 1; lane 7) MIS; and lane 8) Smad 9. It is noted that rhMIS has recently been shown to induce pro-apoptotic effects in several cancer cell lines derived from the female reproductive tract (See Teixeira J, Maheswaran S, Donahoe P K 2001 Müllerian inhibiting substance: an instructive developmental hormone with diagnostic and possible therapeutic applications. Endocr Rev 22:657-674, the contents of which are hereby incorporated by reference). FIGS. 2A-2F: Müllerian-inhibiting substance (MIS) and MIS type II receptor (MISRII) protein are expressed in human endometrium. Fixed sections of human proliferative (A, D) and secretory (B, E) endometrium stained for MIS (A, D) and MISRII (B, E) revealed strong immunoreactivity for both proteins in the glandular cells. Stromal cell immunoreactivity for these proteins appeared to be increased in the secretory phase (B, E). Use of secondary antibody only as a negative control revealed no immunoreactivity (C, F). When the MIS antibody was preadsorbed with fivefold excess recombinant MIS, an absence of immunoreactivity was also noted.

The data disclosed demonstrates that adult human endometrium possesses an intact and functional MIS autocrine/paracrine system whose role appears to be the regulation of cellular proliferation. This laboratory has also identified a role for MIS in endometrial tissue modifications throughout the menstrual cycle. Controlling this intrinsic system for endometriosis treatment is feasible using a more potent and/or longer-acting MIS analogue produced through glycoengineering in which the number of carbohydrate moieties on the native MIS molecule will be significantly increased. Based on the discoveries disclosed herein, MIS is expected to inhibit proliferation or enhance apoptosis in cells derived from both normal endometrium and endometriosis implants given its pro-apoptotic effects in the Müllerian duct as well as in several cancer cell lines in vitro.

The investigation of a long-acting MIS analogue to inhibit the proliferation of ectopic endometrial implants without inducing concomitant hypo-estrogenic side effects associated with traditional hormonal therapies is a breakthrough treatment of endometriosis. The current recommendation for all patients with infertility and suspected endometriosis is to undergo a laparoscopy to diagnose and ablate all endometrial implants (Marcoux S, Maheux R, Berube S 1997 Laparoscopic surgery in infertile women with minimal or mild endometriosis. Canadian Collaborative Group on Endometriosis. N Engl J Med 337:217-222). At the time of such laparoscopy, a potent MIS analogue could be delivered to the site, e.g. in a functionalized hydrogel or in porous PLGA microspheres. The MIS can be applied directly to endometriotic lesions that are known to recur after surgical removal or ablation, such as ovarian endometriomas, or to lesions in locations too precarious to resect or ablate, such as bladder or bowel implants. Local application would eliminate any possible negative effects upon normal endometrium that could potentially arise from systemic administration.

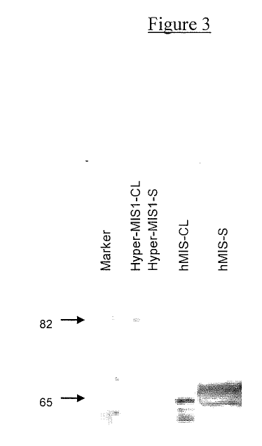

Making a long-acting MIS agonist is a challenging design exercise primarily because MIS is released as a pre-pro-hormone that is cleaved and enzymatically activated at the site of protein action. Thus, one must ensure that the expressed protein possesses the sequence and proper structure to be activated at the site. The aim was to design a form of MIS that would be resistant to degradation in vivo, have a long circulating half-life and be relatively easy to purify. These points were addressed by incorporating into the 3′ end of the modified MIS gene a sequence, N4, (four repeats of an N-linked glycosylation recognition sequence). Also incorporated into the MIS cDNA sequence were two antigenic tags, V5, for protein identification by western blot and hexahistidine, for affinity purification. When this construct was transiently transfected into CHO-K1 cells pro-hyper-MIS1 was identified at the expected molecular weight in the cell lysate fraction but not in the supernatant fraction. This suggested the nascent protein was not being processed for secretion (FIG. 3). This problem was addressed by making an MIS cDNA clone modified to contain a furin protease cleavage site sequence that, when expressed in furin-producing HEK293 cells, is secreted into the supernatant. In addition, stable clones of the modified hyper-MIS2 construct will be prepared for expression in HEK293 cells and protein production.

All study participants gave written informed consent prior to participating in this study, which was approved by the IRB of Columbia University. Approximately 1 cm² pieces of endometrial tissue were obtained from adult women in differing phases of their menstrual cycle at the time of their medically indicated endometrial biopsy, endometrial curettage, or hysteroscopic surgery. These samples were then divided into equal portions for gene expression studies, cell culture, IHC, and routine pathological evaluation.

Immunohistochemistry (IHC) was performed with either antibodies to human MIS or antibodies to MISRII and visualized with Diaminobenzidine tetrachloride (Vector Laboratories, Burlingame, Calif.), which revealed that both MIS and MISRII protein are expressed in normal human proliferative and secretory endometrium, primarily in the glandular epithelium with an increase in stromal expression noted in the secretory phase (data not shown).

Immunocytochemistry of cultured human endometrial glandular and stromal cells indicate that MIS and MISRII protein production are primarily restricted to cells undergoing mitosis. ELISA revealed that MIS is actively secreted by human endometrial stromal cells in vitro. In addition, increasing local MIS concentrations in cultured human endometrial stromal cells both by exogenous administration or transient transfection significantly decreased the number of viable cells and increased their caspase 3/7 activity. These studies confirm that adult human endometrium possesses an intact and functional MIS autocrine/paracrine system whose role appears to be the negative regulation of cellular proliferation.

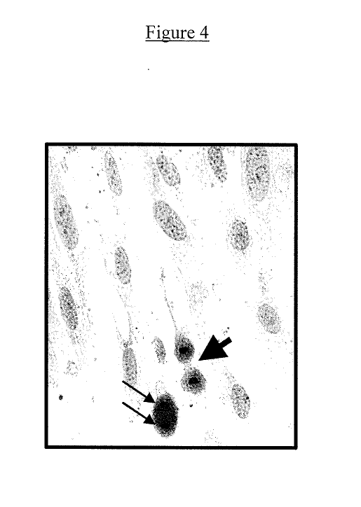

MIS and MISRII Expression During Mitosis in Cultured Endometrium: Human endometrial tissue was cultured. When cells reached 80% confluence, they were fixed and IHC was performed to visualize expression of MIS and MISRII. IHC indicate that MIS and MISRII proteins are produced but are primarily restricted to cells undergoing mitosis (FIG. 4). Interestingly, this finding corroborates those of a prior study demonstrating a similar positive correlation between MIS mRNA levels and mitotic activity in rat ovarian granulosa cells (20). The temporally restricted expression of MIS/MISRII during mitosis strongly suggests this signaling pathway may be an important autocrine/paracrine regulator of endometrial stromal cell division.

MIS Expression in ESC and Estradiol: Based on the above findings, it was hypothesized that estradiol, a known endometrial stromal cell mitogen, would increase the mitotic activity and thereby increase the expression of MIS. Estradiol treatment of ESC significantly increased MIS secretion by 57% (P<0.0001) when compared to that of the vehicle control group using Students t-test. This observation further provides evidence that the MIS signaling pathway may play a critical role in regulating the proliferation of endometrial stromal cells.

Elevated levels of MIS Induces Growth Inhibition in Cultured Endometrium: Given the results from the experiments described above, rhMIS (R&D Systems, Minneapolis, Minn.) was used to investigate the effect of the MIS/MISRII pathway on endometrial stromal cell growth. Cultured endometrial cells were treated with rhMIS (100 ng/mL) for 72 hours and then evaluated for cell viability. Treatment with rhMIS resulted in a 14% (P<0.01) decrease in viable cells when compared to the vehicle control group (data not shown). Follow-up studies in which cultured endometrial cells were transiently transfected with an MIS protein expression construct confirmed that over-expression of MIS decreased cell proliferation by 8% (p<0.0003) and also significantly increased the percentage of cells undergoing apoptosis.

The results from these functional studies clearly demonstrate MIS/MISRII pathway to be a key autocrine/paracrine regulator of endometrial stromal cell growth.

For in vitro confirmation of MIS activity using MA10 cells the following steps are performed. To confirm that hyperglycosylation of MIS enhances protein half-life in the manner previously observed with FSH and VEGF, pharmacokinetic studies are done. Hyper-MIS is expected to provide potent anti-growth/pro-apoptotic activity with eutopic endometrial tissue. One widely accepted model of endometriosis is human implanted endometrial tissue. Prior to mouse studies, a series of in vitro assays with human endometrium is performed to further elucidate the mechanistic pathways by which human cultured endometrium proliferates and invades surrounding tissue. The cultures are exposed to both MIS and hyper-MIS to determine what dose of protein is required to inhibit endometrial cell invasion. Data obtained from the in vitro studies permits evaluation of the ability of hyper-MIS to limit endometrial invasion and spread such as would be expected with mild to moderate endometriosis and to decrease the severity of endometriosis which would provide a novel therapy for late stage endometriosis.

As indicated above, the design of the hyper-MIS1 protein was changed to accommodate the mechanism for activating the protein (hyper-MIS2). For protein production and purification the conditioned culture media is collected and purified on a nickel column to sequester the hexahistidine tagged recombinant hyper-MIS2. The concentrated protein will then be eluted, quantified by Bradford assay, and visualized on a Western blot to assess molecular weight and relative purity. Hyper-MIS2 is expected to exhibit similar properties to endogenous or recombinant MIS protein. To confirm MIS activity of hyper-MIS in vitro MA-10 cells are employed. Cells will be thawed, immediately placed in media and grown in grown in T-75 flasks until they are 80% confluent. The cells will then be trypsinized, harvested, and aliquoted in 96 well plates at a concentration of 10⁴ cells/well. Cells will then be treated for 48 hours with either vehicle, recombinant MIS, hyper-MIS2 or sp600125, an inhibitor of JNK. Cells will then be lysed in Trizol RNA extraction reagent in preparation for real time PCR quantification of the expression of the secondary messenger, Smad 6. The results of hyper-MIS will be compared to the rec-MIS and sp600125.

Pharmacokinetic study: To confirm the half-life of hyper-MIS2 is extended by the alteration of protein glycosylation, pharmacokinetic studies will be performed. Briefly, six male Sprague-Dawley rats (200 g) will be given standard chow diet and water ad libitum in an animal facility for at least 3 days before use. Rats will be housed in temperature-controlled rooms with a 12 hour light/dark cycle. The day before the pharmacokinetic experiment, the right femoral veins of the rats will be catheterized using aseptic technique with sterile silastic cannula (Dow Corning, Midland, Mich., USA) under isoflurane anesthesia. After cannulation, Intramedic PE-50 polyethylene tubing (BD Biosciences, Franklin Lakes, N.J.) will be connected to the cannula and exteriorized through the skin. The skin will be sutured to limit organ exposure for the duration of the bleeds. The cannula will be flushed with 0.9% saline.

Rats will dosed intravenously with 10 mg/kg hyper-MIS2 and rhMIS (R&D, Minneapolis, Minn.) dissolved in sterile 0.9% saline. The rats will be given between 0.5 and 1 ml of each formulation as IV bolus within 5 min. After dosing, serial blood samples (±0.30 ml) will be collected from the cannula at 0, 1, and 30 min, then 1, 2, 4, 6, 12, 24, and 48 h after IV administration, and the cannula will be flushed with 0.9% saline after each collection. After dosing and after each serial blood sampling, two blinded observers will be present to record any visible bleeding or behavioral changes suggesting acute toxicity. Each blood sample will be collected in heparinized tubes (Monoject, Mansfield, Mass.) and following centrifugation, the plasma will be collected and stored at −80° C. until analyzed simultaneously by highly specific MIS ELISA (DSL, Webster, Tex.).

Pharmacokinetic analysis will be performed using data from individual rats for which the mean and standard error of the mean (SEM) will be calculated for each group. The elimination rate constant (KE) will be estimated by linear regression of the blood or plasma concentrations in the log-linear terminal phase. In order to estimate the blood or plasma concentrations (C₀) immediately after MIS treatment dosing, a two-compartmental model will be fitted to the plasma concentration versus time data using software (Version 5.1). The estimated C₀ will then be used with the actual measured plasma concentrations to determine the area under the plasma concentration-time curve (AUC). The AUC_{0-∞} will be calculated using the combined log-linear trapezoidal rule for data from time of dosing to the last measured concentration, plus the quotient of the last measured concentration divided by KE. Non-compartmental pharmacokinetic methods will be used to calculate mean residence time (MRT by dividing AUC_{0-∞} by AUC_{0-∞}), clearance (CL by dividing dose by AUC_{0-∞}) and volume of distribution (Vd_{β} by dividing CL by KE). Based on the cumulative urinary excretion, the fraction excreted in urine (fₑ by dividing the total cumulative amount of rapamycin excreted in urine (ΣXu) by the dose), renal clearance (CLᵣ by multiplying fₑ by CL), hepatic clearance (CL_{hepatic} by subtracting CLᵣ from CL), extraction ratio (ER by dividing CL_{hepatic} by hepatic flow (Q)). The mean hepatic blood flow (Q) is approximately 3.22 lL/h kg. Using a hematocrit in rat of 0.48 will yield a mean hepatic plasma flow of 1.74 l/h kg. Therefore, depending on plasma or blood the correct hepatic flow (Q) needs to be employed. The estimated bioavailability could be calculated utilizing two methods: either by subtracting the ER from 1 or by using the relationships F=Q/(Q+CLᵢₙₜ) and CL_{hepatic}=(Q×CLᵢₙₜ)/(Q+CLᵢₙₜ), been CLᵢₙₜ the intrinsic clearance.

In vitro effects of MIS and hyper-MIS on cultured mouse and human endometrium:

Human endometrial tissue collection: Approximately 2 cm³ pieces of eutopic endometrium will be obtained by endometrial biopsy with a pipelle mini-aspirator from 5 women with (Group A) and 5 women without (Group B) endometriosis between the ages of 21 and 35. Women must have a history of regular 28 day cycles and must be off all hormonal therapy for at least the preceding 3 months. These women will be confirmed non-pregnant by serum and urine pregnancy tests as of no more than one day before sampling, and will also be in the late secretory phase of their menstrual cycle (days 25-27). This time period is chosen because the retrograde flow of viable cells in sloughed secretory endometrium is believed to significantly contribute to the ontogeny of pelvic endometriosis in susceptible women (Garai J, Molnar V, Varga T, Koppan M, Torok A, Bodis J 2006 Endometriosis: harmful survival of an ectopic tissue. Front Biosci 11:595-619, and Sampson J 1927 Peritoneal endometriosis due to menstrual dissemination of endometrial tissue into the peritoneal cavity. Am J Obstet Gynecol 14:442-469). A sample from each of the biopsy specimens will be fixed in 4% buffered formaldehyde and embedded in paraffin for histologic examination. For the purposes of this study, only results derived from experiments utilizing human luteal endometrium confirmed to be histologically normal and in-phase will be included in later statistical analyses.

The nude mouse model has been extensively studied and for the most part has served quite useful in helping to understand the physiology and prevention of ectopic endometrial implantation and proliferation. The nude mouse model of endometriosis will be created as previously described (See Grummer R 2006 Animal models in endometriosis research. Hum Reprod Update 12:641-649, the contents of which are hereby incorporated by reference). Ninety nude (Swiss nu/nu) 10 week old female mice (Charles River Laboratories, USA) will be used for the study. One week prior to transplantation, the mice will be ovariectomized to remove the endogenous source of murine MIS which has approximately 70% homology to the human form. Concurrently, sterile 60-day release pellets, containing 1.7 mg of 17-β estradiol (Innovative Research of America; Sarasota, Fla.), will be implanted SC. The mice will be anesthetized with isoflurane (nasal cone) prior to and during the entire procedure. After sterilizing the skin with 95% ethanol, an ip injection of the homogenized endometrial tissue with a thin 19 G needle will be made. The endometrial tissue cell suspension prepared from one patient will be divided among 9 mice (one study group).

Eutopic endometrium from each of the 10 patients will be prepared as follows: Initially it will be placed into DMEM/F12 media with 10% antibiotics, mechanically cleared of clots and debris, minced into microfragments able to pass through a 19 gauge needle, and incubated in a solution of 8 μmol/l of carboxyfluororescein diacetate, succinimidyl ester (CFDA SE) (Molecular Probes, USA) in PBS for 15 minutes in a 37° C. water bath. Labeling will be ensured by visualization under fluorescence microscopy and viability will be checked by the Trypan blue dye exclusion. The skin will be sutured with a single 3-0 vicryl stitch.

After allowing the mice to recuperate for 3 days, during which time their endometriosis will establish itself, three mice from each group of nine mice receiving endometrial transplants from a single patient will be randomized to receive twice daily ip injection of 100 μg rhMIS (R&D Systems); three will receive twice daily ip injections of 100 ug rhMISa; the remaining three will receive saline vehicle injections. This protocol of rhMIS administration in adult female CH3 mice results in circulating levels of 2-4 μg/mL (14-28 nM) (See Segev D L, Hoshiya Y, Stephen A E, Hoshiya M, Tran T T, MacLaughlin D T, Donahoe P K, Maheswaran S 2001 Müllerian inhibiting substance regulates NFkappaB signaling and growth of mammary epithelial cells in vivo. J Biol Chem 276:26799-26806, the contents of which are hereby incorporated by reference). In total, thirty mice will receive rhMIS (5 from Group A endometrium in triplicates +5 from Group B endometrium in triplicates), thirty mice will receive MIS analogue (5 from Group A endometrium in triplicates +5 from Group B endometrium in triplicates), and thirty mice will receive saline injections.

After 5 days, the mice will be euthanized by CO₂ asphyxiation followed by cervical dislocation. Whole blood will be collected by cardiac puncture and frozen for later analysis of serum MIS levels. Implanted endometrial lesions will be recovered by laparotomy as macroscopic lesions are recognized by their nodular aspect distinct from murine tissues. Further dissection under an inverted fluorescence microscope will aid the identification of microscopic lesions. The ovaries and a portion of the uterus will be fixed in 4% buffered formaldehyde and embedded in paraffin for histologic evaluation.

Endometriosis-like lesions will be quantified by measuring the fluorescence of CFDA SE at λ^{ex465}/λ^{em535} using a Kodak 2000MM image station. The sum of the background-subtracted pixel values within the region of interest will be obtained using the Kodak 1D image analysis software. After the fluorimetric analysis, a portion of the specimen will be store in RIPA for the confirmation of MISRII expression by Western Blot analysis. The remaining portion will be placed in 4% buffered formaldehyde and embedded in paraffin for histologic evaluation.

The paraffin embedded lesions will be cut into 5 μm sections and every fifth slide stained by haematoxylin-eosin. The two sections surrounding the largest endometriotic lesion surface will be selected for IHC staining Briefly, immunostaining will be performed by incubating the specimens with the following three primary antibodies overnight at 4° C.: 1) mouse monoclonal antibody to human CD 10, a marker for endometrial stroma (clone 56C6, Novocastra, Newcastle, UK), 2) mouse monoclonal antibodies to human cytokeratin cocktail CK22, a marker for endometrial epithelium (Biomeda, USA), and 3) rabbit antibody to human Ki67 for proliferation activity (Dakocytomation). Following incubation with the appropriate secondary antibody conjugated to peroxidase, 3,3′-diaminobenzidine will be used to reveal the presence of peroxidase. Surface areas of the glandular and stromal structures of the lesions will be measured on the CD10- and CK-22 stained slides with an image analysis system. Proliferative activity will be evaluated by counting the number of Ki67-positive and -negative nuclei of epithelial cells in each lesion. The proliferative activity of stromal cells has been consistently demonstrated to be <1% in prior studies using similar murine models of endometriosis and therefore will not be analyzed in this study (See Nisolle M, Casanas-Roux F, Marbaix E, Jadoul P, Donnez J 2000 Transplantation of cultured explants of human endometrium into nude mice. Hum Reprod 15:572-577, and Van Langendonckt A, Eggermont J, Casanas-Roux F, Scholtes H E, Donnez J 2004 Expression of platelet endothelial cell adhesion molecule-1 in red and black endometriotic lesions. Feral Steril 82:984-985, the contents of which are hereby incorporated by reference). As compared to both saline vehicle and rhMIS, it is expected that twice daily i.p. injections of MISa will result in significantly less lesions recovered, less endometriosis-like lesions as measured by CFDA SE fluorescence and by IHC, and a lower proliferative index as measured by Ki67.

Statistical Analysis: The effect of treatment with rhMIS, MISa, or saline vehicle control on the growth of eutopic endometrial transplanted into nude mice will be compared using the number of established lesions, the total amount of recovered lesions estimated by the fluorescence of CFDA SE and by the lesion size on CD-10 and CK-22 stained slides, and the proliferative index measured by Ki67. Each parameter will be estimated by the mean value from the set of triplicates exposed to the same experimental conditions. Standard deviations and the coefficient of variation will be calculated to assess the reliability of the model. The values for each measure among all 3 groups will be compared using one-way ANOVA. Post-hoc comparisons between groups will be performed by Tukey-Kramer HSD method. (Non-parametric data would be initially assessed by Kruskal-Wallis analysis of ranks.)

What is claimed is:

1. A Müllerian Inhibiting Substance (MIS) analogue comprising one or more non-naturally occurring N-linked glycosylation recognition sequences and an approximately 108-109 amino acid C-terminal fragment which can be released from the MIS analogue by proteolytic cleavage, wherein the N-linked glycosylation recognition sequences are located outside of and N-terminal to the C-terminal fragment.

2. The MIS analogue of claim 1, wherein the MIS analogue comprises four to six N-linked glycosylation recognition sequences.

3. The MIS analogue of claim 1, wherein the MIS analogue further comprises a non-naturally occurring protease cleavage site located within the five amino acids N-terminal to the C-terminal fragment.

4. The MIS analogue of claim 1, wherein the protease cleavage site is a furin cleavage site.

5. The MIS analogue of claim 1, wherein the MIS analogue further comprises a tag sequence selected from the group consisting of myc, T7, GST, GFP, HA (hemagglutinin), V5, His, GST, and FLAG tags.

6. The MIS analogue of claim 1, wherein the MIS analogue comprises a sequence that has about 90% or greater sequence identity with SEQ ID NO. 1.

7. The MIS analogue of claim 1, wherein the MIS analogue comprises SEQ ID NO. 1.

8. A pharmaceutical composition comprising the MIS analogue of claim 1 and a pharmaceutically acceptable carrier.

9. A method for inhibiting proliferation of, or inducing apoptosis in, cells derived from the Müllerian duct, the method comprising contacting cells derived from the Müllerian duct with an effective amount of the MIS analogue of claim 1.

10. The method of claim 9, wherein the cells are in vitro.

11. The method of claim 9, wherein the cells are in a human female subject.

12. The method of claim 9, wherein the cells derived from the Müllerian duct are selected from the group consisting of endometrial cells, other uterine cells, ovarian cells, cells of the oviduct, cervical cells, and vaginal cells.

13. A method for treating a condition associated with uncontrolled growth of a tissue derived from a Müllerian duct in a subject, the method comprising administering to the subject an effective amount of the MIS analogue of claim 1.

14. The method of claim 13, wherein the subject is a human female.

15. The method of claim 14, wherein the human female has or is at risk of developing a condition selected from the group consisting of endometriosis, adenomyosis, uterine fibroids, cancer of the cervix, cancer of the ovary, and cancer of the uterus.

16. A method for treating endometriosis in a human female subject, the method comprising administering to the subject an effective amount of a Müllerian Inhibiting Substance (MIS) analogue of claim 1.
